(19) United States
(12) Patent Application Publication
(10) Pub. No.: US 2006/0172407 A1
(43) Pub. Date: Aug. 3, 2006

(54) Bacterial cell digestion

(76) Inventors: Frank Mayer, Bovenden (DE); Andreas Schwienhorst, Goertingen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/520,145

(22) PCT Filed: Jul. 2, 2003
(86) PCT No.: PCT/EP03/07068

§ 371 Date: Nov. 7, 2005

(30) Foreign Application Priority Data
Jul. 2, 2002 (DE) 102 29 645.9

(51) Int. Cl. C12N 9/48; C12N 1/06

(52) U.S. Cl. 435/259; 435/212

(57) ABSTRACT

The invention relates to the use of substances which bind to the bacterial elongation factor EF-Tru for cell digestion or for the lysis of cells. The invention further relates to a cell digestion system and a cell digestion method.

The invention relates to the use of substances that bind to the bacterial elongation factor EF-Tu for cell digestion or for lysis of cells. Furthermore, the invention relates to a cell digestion system or a cell digestion process.

Cells, especially bacteria cells, are often used to produce compounds using molecular-genetic techniques, for example by homologous or heterologous gene expression and expression of synthetically active enzymes. Preferably peptide compounds, triglycerides, waxy esters and PHAs and especially those compounds that can be used in biotechnology, medicine and the pharmacy sector, or in other areas, are produced. In the case of heterologous gene expression, the compounds formed are non-natural components of the cells used or the bacteria used. The production of the most varied compounds by means of gene expression is extensively described in the prior art (see, e.g., Q. Bi et al., Applied Biochemistry and Biotechnology, 95(1) (2001) 23-30; D. Macmillan et al., Chemistry and Biology, 8(2) (2001) 133-45; J. E. de Oliveira et al., Journal of Chromatography, A, 852(2) (1999) 441-50 and M. Schmidt et al., Journal of Biotechnology, 68(1) (1999), 71-83). In this way, for example, recombinant human erythropoietin, recombinant human insulin and recombinant human growth hormone were produced.

Recovering the compounds produced in the cells, especially when they are substances that have been synthesized intracellularly, generally dictates that the cells or bacteria must be lysed. Such a lysis is always imperative when the desired compounds cannot be slipped out of the living cell. In this case, the desired compounds can only be released by lysis and can be supplied to further processing (down-stream processing).

In order to produce cell lysis, for example enzymes, such as, for example, lysozyme, can be added; they lyse the cell coating, lysis not being attainable by induced lysozyme expression. When the cell coating is destroyed, the cell can be considered “digested” (T. R. Hopkins, Bioprocess Technology (New York) 12 (1991), 57-83; J. A. Asenjo et al., Bioprocess Technology (New York) 9 (1990), 143-75). Processes can also be used for lysis in which mechanical shearing forces act to destroy the cells. One example of such a process is the use of a French press.

The digestion of bacteria in biotechnological production processes represents a major cost factor. Moreover, in the known cell digestion process, disruptive side effects often occur, such as, for example, strong proteolysis or excessive presence of cellular debris.

Therefore, the object of this invention was to devise an improved process for cell digestion, especially a process that can be advantageously used in biotechnological production processes.

This object is achieved according to the invention by the use of substances that bind to the components of the cytoskeleton, especially to EF-Tu, for cell digestion.

The discovery of the existence of a bacterial cytoskeleton and the characterization of its components, especially the characterization of the elongation factor EF-Tu, as a structurally important protein for this cytoskeleton, open the door to a new system for cell digestion, especially cell digestion of bacteria. Thus, according to the invention, an alternative to conventional cell digestion processes is made available. By destabilizing the cytoskeleton, the cell coating can be destroyed and thus lysis of the cells can be induced.

Preferably, according to the invention, to destabilize the cytoskeleton, substances are used that bind to EF-Tu.

The bacterial protein EF-Tu contains domains 1, 2 and 3 (H. Song et al., J. Mol. Biol. 285 (1999) 1245-1256). The sequences of the EF-Tu protein and its coding gene in Escherichia coli and a series of other Eu bacteria are published and are accessible in databases. EF-Tu is a protein that contains three domains. Of the 394 amino acids that EF-Tu (of E. coli) comprises, amino acids 8 to 204 belong to domain 1, amino acids 172 to 204 forming a connecting structure to domain 2. Amino acids 205 to 298 belong to domain 2 and amino acids 299 to 394 belong to domain 3.

According to the invention, it was found, surprisingly enough, that in the bacterial cytoskeleton, destabilization and consequently lysis of cells can be achieved by inhibition of the self-assembly of EF-Tu fibrillae.

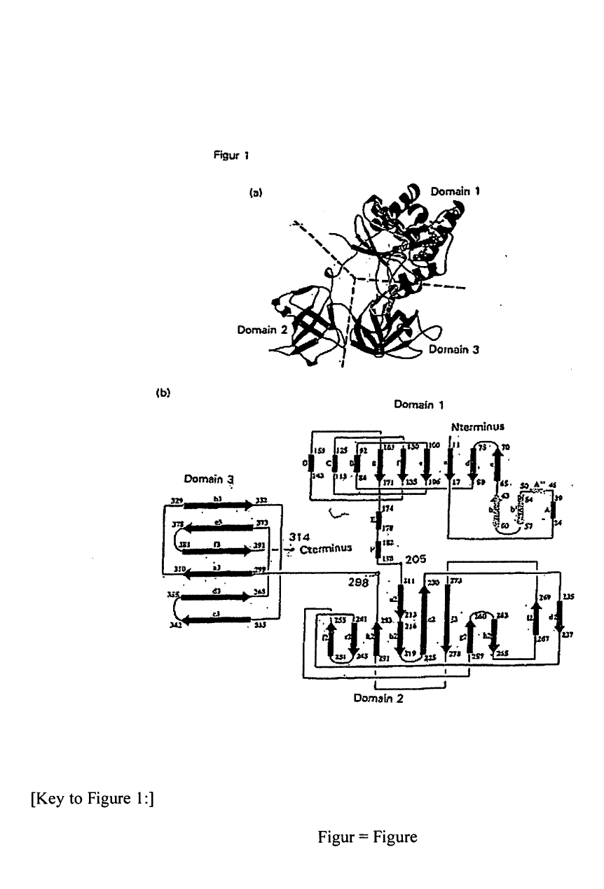

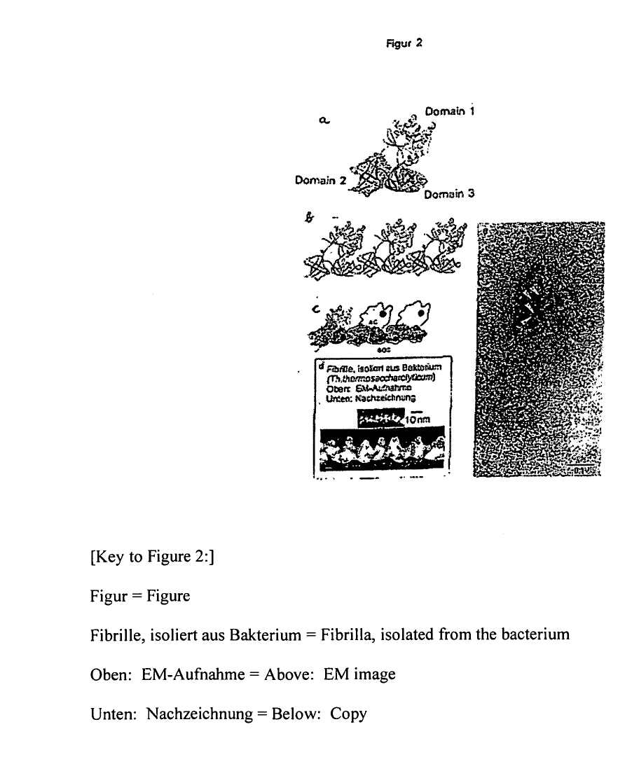

EF-Tu is a 3-domain protein, as shown in FIG. 1. The protofilaments of the cytoskeleton are formed in living cells by domains 2 and 3 of EF-Tu proteins (monomers or integrated into a protofilament and only transiently free) interacting with the domains 3 and 2 of adjacent EF-Tu proteins. By formation of specific non-covalent bonds, the domain 2 of one EF-Tu interacting with the domain 3 of the adjacent EF-Tu, during a self-assembly process, chains form in this way, so-called protofilaments that are joined into a network (cf. FIG. 2). Other factors can be associated with this network, such as, e.g., FtsZ, MreB and/or M6l.

Stability and/or development of these protofilaments and networks in bacteria cells can be prevented according to the invention, as a result the death of the bacteria cells by lysis occurring.

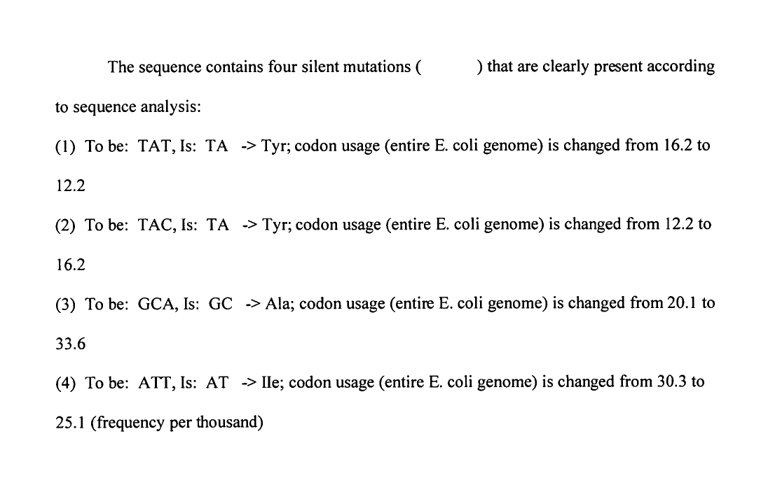

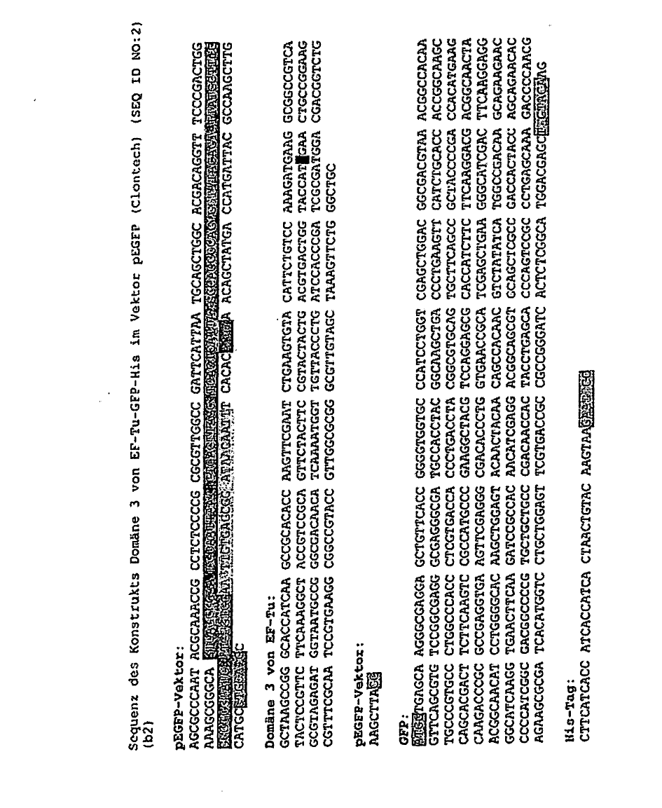

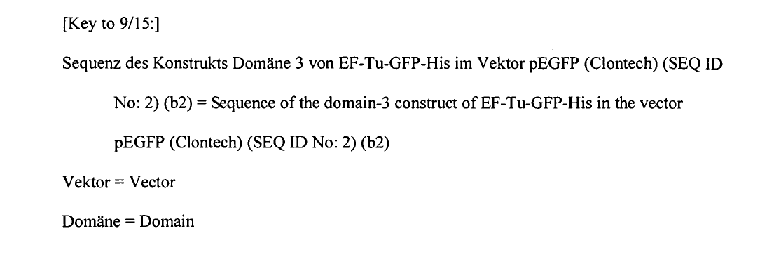

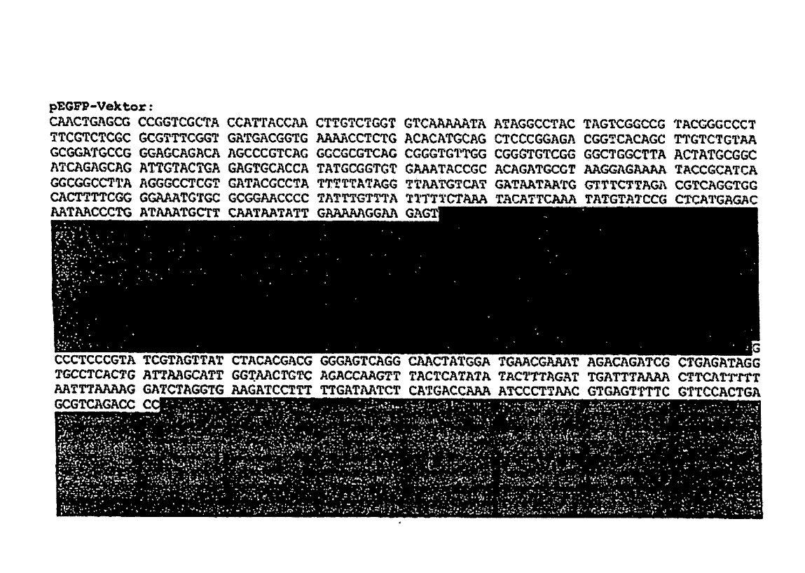

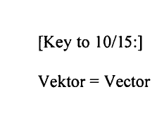

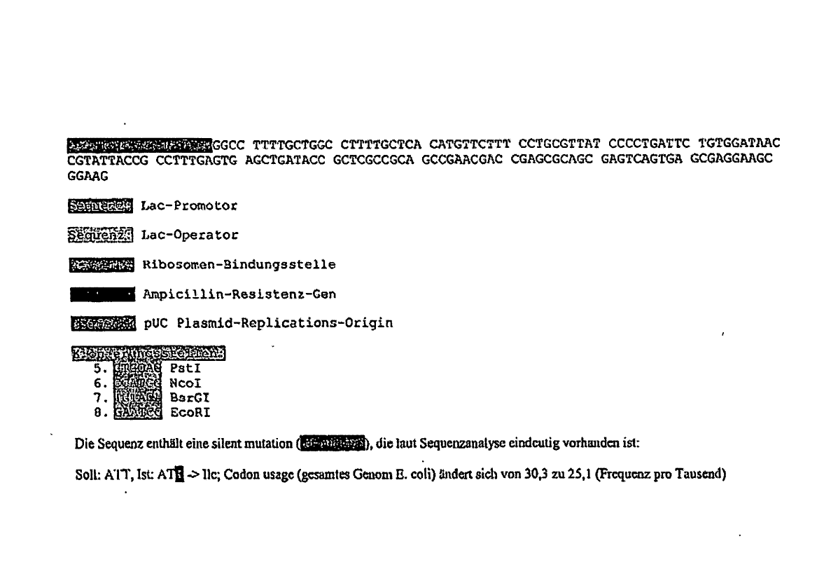

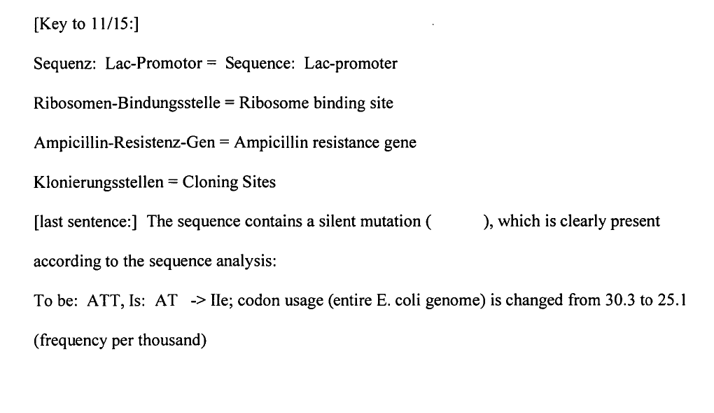

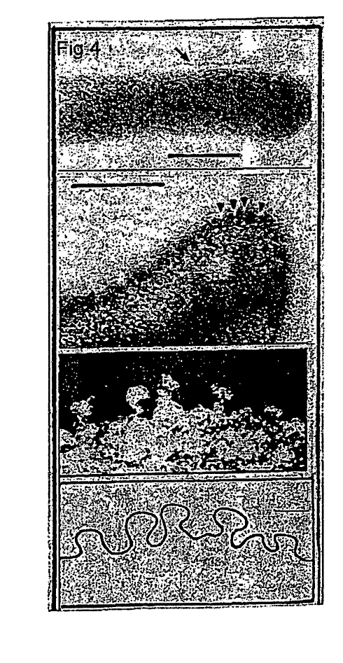

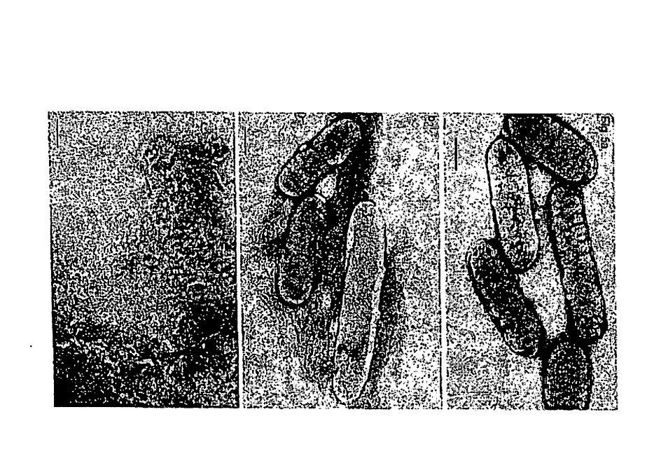

One especially advantageous procedure of the invention is as follows. The DNA segment of the EF-Tu gene of Escherichia coli that codes for the domain 3 is obtained and by means of molecular-genetic techniques is transferred into E. coli cells and expressed. In addition to this recombinant segment that contains solely the domain 3, but not domains 1 and 2 of EF-Tu, the cells as before have the native EF-Tu gene and can express it. The additionally synthesized domain 3 polypeptides, however, compete with the native EF-Tu proteins around the binding sites for the formation of the protofilaments. If a domain-3 polypeptide instead of a native EF-Tu protein is incorporated as a link, this leads to chain rupture as a result of the absence of the second binding site (the domain 2 is essential for chain formation) on the domain-3 polypeptide. The protofilament does not continue to grow, and the cytoskeletal network is weakened and finally collapses. As a result of the loss of integrity of the cytoskeleton, the cytoplasmic membrane of the cell loses it suspension. The cytoplasmic membrane is, as shown in FIG. 4, suspended on the cytoskeleton. In this way, the cell wall that in the living bacteria cell is positioned and supported by the cytoplasmic membrane is destroyed (see FIG. 5). The loss of the cell wall and the cytoplasmic membrane means exposure of the contents of the cell so that the cell is lysed (cf. FIGS. 5 and 6). The cell contents can thus be recovered without further cell digestion.

Cells that are used for biotechnological production processes, such as, for example, heterologous expression, production of proteins, especially for biotechnological or medical purposes, can thus be digested by introducing into them, before their use for production, a sequence that codes for the domain of the EF-Tu that correspond to the cell species.

In one preferred embodiment, the substances used for cell digestion contain portions that bind to EF-Tu in the area of amino acids 218 to 224 of the domain 2 and/or in the area of the amino acids 317 to 328 and/or 343 to 354 of the domain 3. Within domains 2 and 3, different secondary structures occur. In this case, the amino acid sequences from 317 to 328 and from 343 to 354 that lie in the domain 3 and form loops are of special interest; the loops project freely into space and are candidate sequences for interaction with amino acid sequences that lie in a correspondingly positioned dent on the periphery of the domain 2, these sequences extending from amino acids 218 to 224.

In one preferred embodiment of the invention, intracellularly expressed peptide substances are used that are based on oligopeptides that bind to EF-Tu, preferably in the area of the fitting sites of domains 2 and/or 3. These oligopeptides can contain partial segments of amino acid sequences of domains 2 and/or 3 with a length of preferably 4 to preferably 20 amino acids, in particular preferably 5 to 15 amino acids, and especially preferably with a length from 6 to 12 amino acids.

In one especially preferred embodiment, the substances contain partial segments or the entire region of the amino acid sequences from the domain 3 with a length of at least 4 and especially at least 5 amino acids, which at the same time does not correspond to any segment of the amino acid sequences from the domain 2.

In addition to intracellularly expressed peptide compounds, intracellularly expressed peptide mimetic agents can also be advantageously used.

A further subject of the invention is a process for digestion of cells in which components of the cytoskeleton in the cells are destabilized. Cytoskeletal elements that can be changed to cause weakening of a cytoskeleton comprise, for example, FtsZ, MReB, Mbl and contractile proteins and especially EF-Tu.

Especially preferably substances are used in this process that bind to components of the cytoskeleton, especially to EF-Tu, and are explained previously herein.

The invention can be used especially advantageously in a process for producing a compound, for example a protein, by using cells in which this protein is expressed, for example, by a biotechnological production process and by introducing into these cells beforehand a sequence that codes for a substance that destabilizes components of the cytoskeleton of the cells. The lysis that is ordinarily necessary in conventional biotechnological process management for obtaining the compounds formed in bacteria that have been manipulated by genetic engineering can be carried out here with the cell digestion that is described herein. It is advantageous that cell digestion can be carried out at an exactly defined instant. One indicator of this can be achieving the desired cell density (quorum sensing). Induction of lysis by quorum sensing takes place by the cell culture being monitored in its growth behavior and when it has been established that the desired state of the cell culture has been reached, the introduced gene is expressed.

In one preferred embodiment, a construct is introduced into the cells; this construct contains, in addition to the gene for the destabilizing substance, other coding sequences that make it possible to induce the start of synthesis of the substances, for example of the domain 3 polypeptide, from the outside. In this way, it is possible to exactly control the instant of cell lysis in the cell culture from the outside.

Induction by a quorum-coupled and/or cell culture-self-sufficient process is another suitable form for initiating cell lysis.

The invention furthermore comprises a construct, comprising a sequence that codes for a substance that destabilizes components of the cytoskeleton of cells. This construct furthermore preferably comprises at least one gene segment that allows the induction of synthesis of the substance that destabilizes the cytoskeleton.

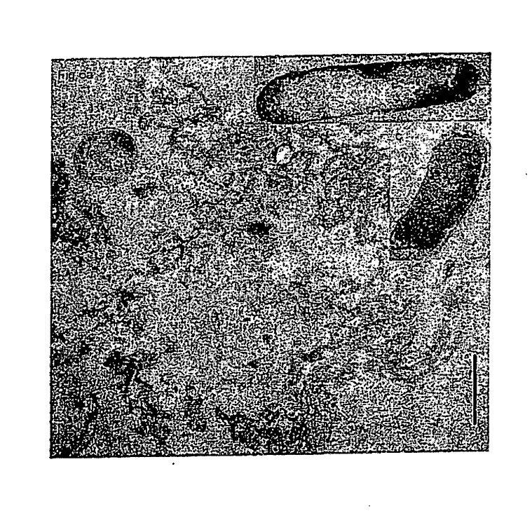

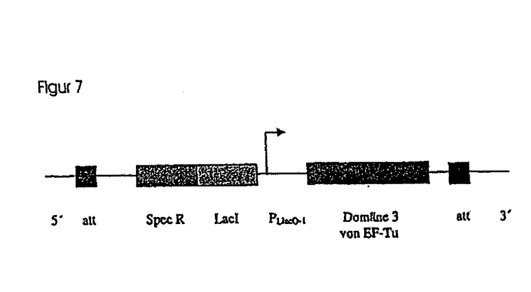

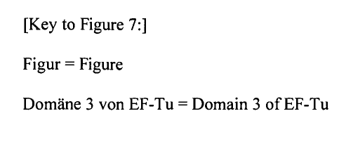

Advantageous layouts of the construct are shown in FIG. 7. As soon as induction has taken place, within one cell generation, lysis of the cells is induced by the interplay of EF-Tu, the domain-3 polypeptide, the cytoplasmic membrane and the cell wall.

The invention is further explained by the attached figures and the following examples:

In the figures,

FIG. 1 shows:
a) EF-Tu (GDP form from E. coli; result of x-ray structural analysis; figure taken from the literature and modified). The 3 domains of the protein molecule are labeled. A′ and b refer to connection sequences
b) Like a), but another form of the representation. The numbers label the positions of the amino acid residue; the N-terminus and C-terminus are indicated.

FIG. 2 shows:
a) EF-Tu molecule
b) and c) The principle of “chain formation” (self-assembly of EF-Tu monomers into protofilaments). The binding takes place by contact between the domains 2 and 3 of adjacent molecules
d) Electron microscope image of such a protofilament, isolated from the bacterium Thermoanaerobacterium thermosaccharolyticum
e) Network, consisting of protofilaments. Electron microscope picture of a network isolated from the bacterium Th. thermosaccharolyticum. The black points are gold markers used for labeling purposes.

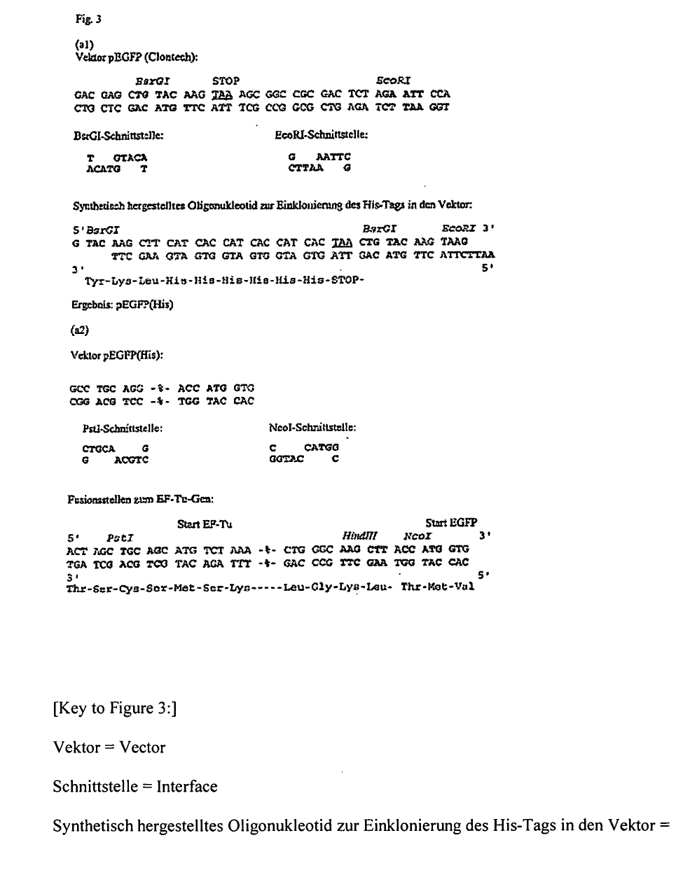

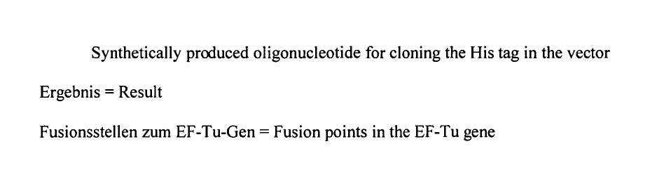

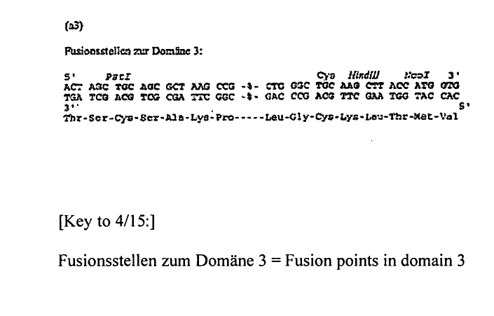

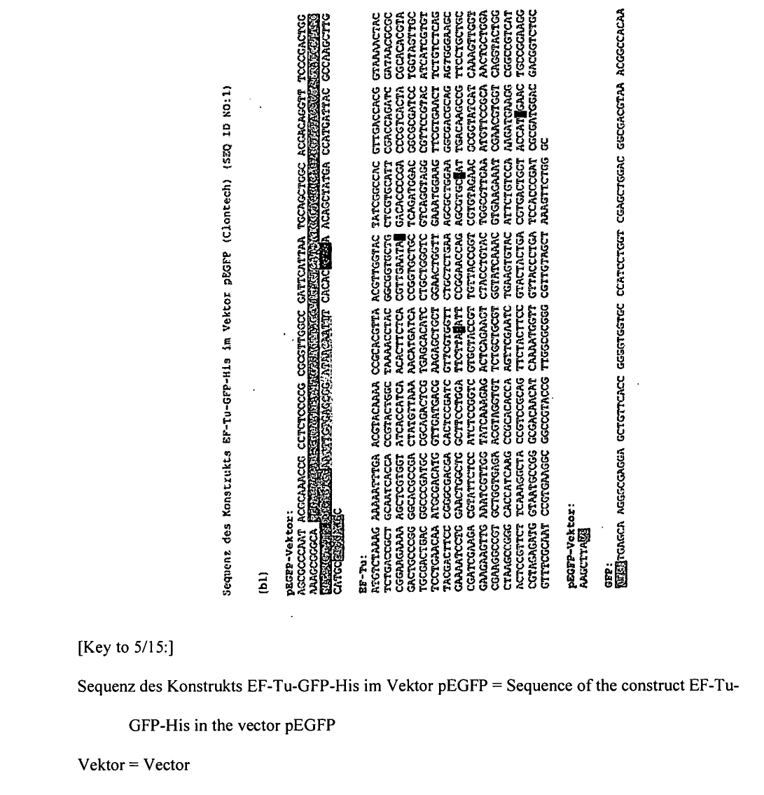

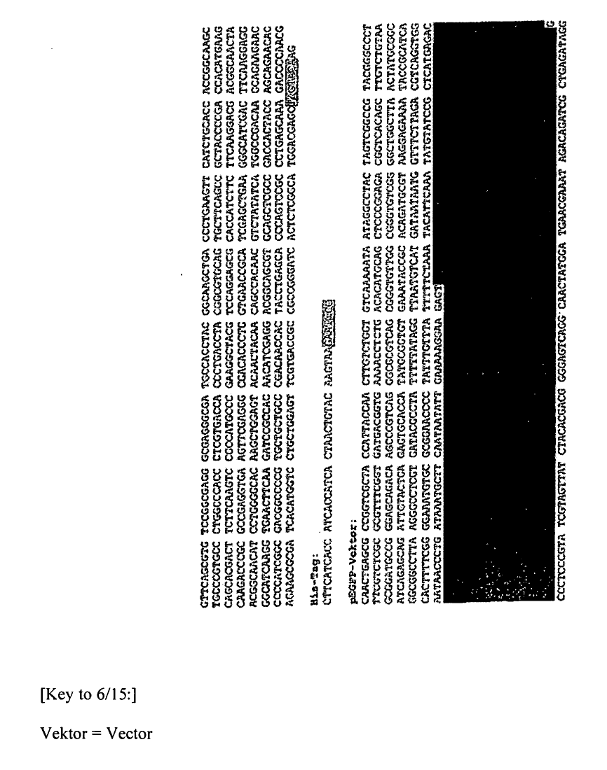

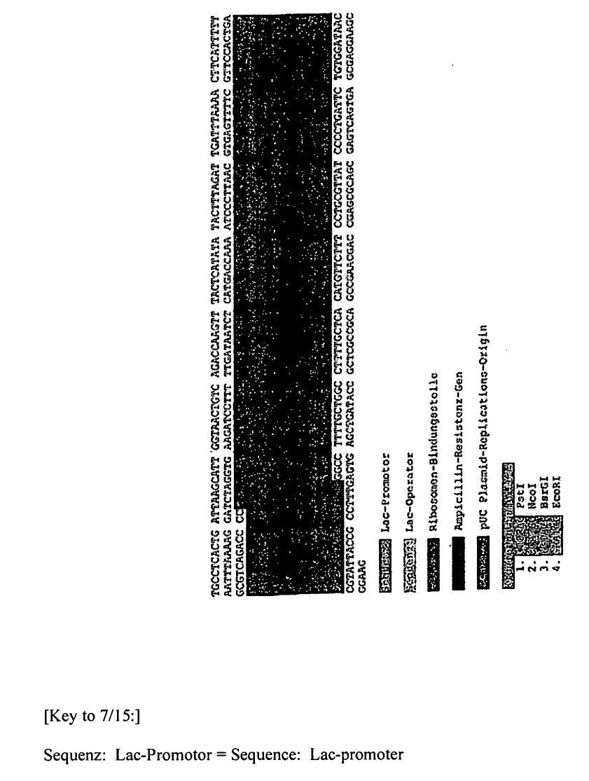

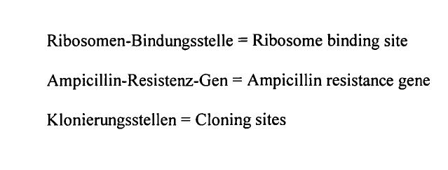

FIG. 3 shows:
EF-Tu-GFP-His fusion clones are to be generated. The ideal fusion point in EF-Tu is the C-terminus that projects between the domain 2 and 3 (Song et al., 1999). The same fusion point can also be chosen for a domain-3 fusion construct. The domain-3 fusion construct can be used for in-vivo competition experiments. Here, it seems appropriate to introduce a C-terminal cysteine (the only one in domain 3) for chemical labeling alternatives.

The starting point is genomic DNA of an E-coli-K-12 strain. With respect to possible later experiments in vitro, it seems appropriate to also provide all constructs with an His tag.
(a1) Introduction of an His tag into the BsrGI/EcoRI-interfaces of the vector pEGFP
(a2) Introduction of the EF-Tu gene into the HindIII/NcoI-interfaces of the vector pEGFP(His)
(a3) Introduction of the domain-3 gene segment into the HindIII/NcoI-interfaces of the vector pEGFP(His)
(b1) Base sequence of the construct pEGFP-EF-Tu-His on the example of the clone HE1 (SEQ ID NO:1)
(b2) Base sequence of the construct pEGFP-domain3-His on the example of the clone HD1 (SEQ ID NO:2)

FIG. 4 shows:
“Suspension” of the cytoplasmic membrane (CM) in the wall-less bacterium Mycoplasma pneumoniae. Electron microscope pictures.
a) Untreated bacterium. The arrow points to the CM
b), c), d) After detachment of the CM (by a detergent), it becomes evident that the CM was suspended on supports (“stalks”) that for their part sit on the peripheral part of the cytoskeleton. If this part of the cytoskeleton is weakened or destroyed, the “stalks” lose their fixed anchoring, and the CM collapses.

FIG. 5 shows:

Tests with E. coli, Electron microscope pictures, negative contrasting
a) Intact bacteria cells, containing their chromosome and in addition an empty vector (plasmid) that was used in continuing tests as a vector for introducing the domain-3 gene segment. These cells therefore represent controls.
b) Cells of a young culture. A plasmid that in expressible form contains the gene segment that codes for the domain 3 of EF-Tu had been introduced into these cells before the start of cultivation, in addition to its own native chromosome (that does contain the gene that codes intact EF-Tu). The effect of the expression of this “truncated” gene can be clearly seen: In addition to some cells that appear intact (they are recognizable as already weakened in their cell coating with a more exact analysis), the picture is determined by cell residues that indicate the original cell form, but have neither a wall nor CM. The wall and the CM have lost their suspension by destabilization of the cytoskeleton. The cell contents are exposed. Such a state is generally referred to as “digested.”
c) End stage of the dissolution of the cells (old culture). The onetime cell form can no longer be detected; the cell residues form unorganized aggregations

FIG. 6 shows:

Tests with E.coli. Electron microscope pictures
a) End stage of the dissolution of the cells of a culture in which the domain 3 of EF-Tu was expressed in addition to the cell's own EF-Tu; ultrathin section (cf. FIGS. 5 b) and c))
b) Control: Cell with empty vector; the cell is intact (cf. Figure 5a))
c) Control: In the cell, in addition to the cell's own EF-Tu, EF-Tu that has been introduced by genetic engineering has still been expressed; the cell is intact

FIG. 7 shows:

Chromosomal integration of a possible construct of the domain 3 of EF-Tu with controllable promoter and resistance marker with the aid of the attachment sites of the phage lambda. The promoter and resistance can also be used in other combinations as required.
Att attachment sites
Spec R spectinomycin resistance
Lac1 repressor
P_{Llac0-1} modified Lac promotor (according to LUTZ and BUJARD, 1997)
As alternatives to the indicated promoter, for example, also the promoters ara, T7 or bdA (quorum sensing) can be used.

### EXAMPLES

### Example 1

### Progression from Cloning to Expression and Purification of the Construct EF-Tu-His or Domain 3-His (shown Schematically):

1. Preparation of genomic DNA of E. coli XL1-Blue (K12 derivative)
2. PCR with the corresponding primers for high concentration of the full-length products
3. Identification and purification of the PCR full-length products
4. Restriction digestion of PCR products and of the target vector
5. Ligation of the PCR products into the target vector
6. Electroporation of the ligated plasmid DNA into the strain XL1-Blue
7. Identification of the clones with the correct insert by restriction digestion and sequence analysis
8. Safeguarding of the clones as cryocultures
9. Plating of the clones out onto selection agar (here: LB/ampicillin) and culture overnight at 37° C.
10. Inoculation of a 50 ml LB/ampicillin liquid culture, incubation overnight at 37° C.
11. Inoculation of a 11 LB/ampicillin liquid culture, incubation at 37° C. up to OD600 from roughly 0.5 to 0.7, induction with IPTG for at least 4 hours
12. Cell harvest
13. Homogenization by pressure/ultrasound
14. Centrifuging off the cellular debris
15. Purification of the cell lysate that has been clarified in this way via IMAC columns
IPTG isopropyl thiogalactoside
IMAC ion metal affinity chromatography

1. Use of substances that bind to the components of the cytoskeleton, especially to EF-Tu, for cell digestion.

2. Use according to claim 1 for cell digestion of bacteria cells.

3. Use according to claim 1, characterized in that the substances bind to the EF-Tu in the area of the domain 2 (amino acids 205 to 298) and/or of the domain 3 (amino acids 299 to 349).

4. Use according to claim 1, wherein the substances bind to the EF-Tu in the area of the amino acids 218 to 224 of the domain 2 and/or in the area of amino acids 317 to 328 and/or 343 to 354 of the domain 3.

5. Use according to claim 1, wherein the substances contain partial segments of the amino acid sequences from the domains 2 and/or 3 with a length of at least four amino acids.

6. Use according to claim 5, wherein the partial segments have a length from 5 to 15 amino acids, especially from 6 to 12 amino acids.

7. Use according to claim 1, wherein the substances contain the domain 3 of EF-Tu and no other domain of EF-Tu.

8. Use according to claim 1, wherein the substances are chosen from linear or cyclic peptide compounds or peptide mimetic agents.

9. Process for digestion of cells, wherein components of the cytoskeleton are destabilized in the cells.

10. Process according to claim 9, wherein substances that bind to the components of the cytoskeleton are used for destabilization.

11. Process according to claim 9, wherein substances that bind to EF-Tu are used.

12. Process according to claim 9, wherein substances are used that bind to the EF-Tu in the area of the domain 2 (amino acids 205 to 298) and/or domain 3 (amino acids 299 to 394), especially in the area of amino acids 218 to 224 of the domain 2 and/or in the area of amino acids 317 to 328 and/or 343 to 354 of the domain 3.

13. Process according to claim 12, wherein substances are used that contain partial segments of the amino acid sequences from the domains 2 and/or 3 with a length of at least 4 amino acids, especially of at least 5 amino acids.

14. Process according to claim 10, wherein nucleic acids are introduced into the cells that code for the substances that destabilize the cytoskeleton.

15. Process for producing a compound, wherein cells are used into which a sequence has been introduced, coding for a compound that destabilizes components of the cytoskeleton of the cells, the cells are cultivated and in this way the desired intracellular compound is obtained.

16. Process according to claim 15, wherein the desired compound is intracellularly produced by cultivation of cells and, in a second step, lysis of the cells is caused by induction of expression of the compound that destabilizes the cytoskeleton.

17. Process according to claim 16, wherein the desired compound is formed by heterologous expression.

18. Process according to claim 16, wherein the desired compound is formed by homologous expression.

19. Process according to claim 16, wherein induction takes place by quorum sensing.

20. Process according to claim 16, wherein the sequence that codes for a compound that destabilizes the cytoskeleton of the cells in a construct is introduced into the cells, the construct containing additional regions that allow an induction of the synthesis of the compound.

21. Construct, comprising a sequence that codes for a compound that destabilizes components of the cytoskeleton of cells.

22. Construct according to claim 21, furthermore comprising a gene segment that allows the induction of synthesis of the compound that destabilizes the cytoskeleton.


SEQUENCE LISTING (14 sequences)

SEQ ID NO: 1 - DNA, 4527 bp, Artificial Sequence
  Description of Artificial Sequence EF-Tu-GFP-His of vector pEGFP
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagcatgtctaaagaaaaatttgaacgtacaaaaccgcacgttaacgttggtactatcggccacgttgaccacggtaaaactactctgaccgctgcaatcaccaccgtactggctaaaacctacggcggtgctgctcgtgcattcgaccagatcgataacgcgccggaagaaaaagctcgtggtatcaccatcaacacttctcacgttgaatacgacaccccgacccgtcactacgcacacgtagactgcccggggcacgccgactatgttaaaaacatgatcaccggtgctgctcagatggacggcgcgatcctggtagttgctgcgactgacggcccgatgccgcagactcgtgagcacatcctgctgggtcgtcaggtaggcgttccgtacatcatcgtgttcctgaacaaatgcgacatggttgatgacgaagagctgctggaactggttgaaatggaagttcgtgaacttctgtctcagtacgacttcccgggcgacgacactccgatcgttcgtggttctgctctgaaagcgctggaaggcgacgcagagtgggaagcgaaaatcctggaactggctggcttcctggattcttatattccggaaccagagcgtgcgattgacaagccgttcctgctgccgatcgaagacgtattctccatctccggtcgtggtaccgttgttaccggtcgtgtagaacgcggtatcatcaaagttggtgaagaagttgaaatcgttggtatcaaagagactcagaagtctacctgtactggcgttgaaatgttccgcaaactgctggacgaaggccgtgctggtgagaacgtaggtgttctgctgcgtggtatcaaacgtgaagaaatcgaacgtggtcaggtactggctaagccgggcaccatcaagccgcacaccaagttcgaatctgaagtgtacattctgtccaaagatgaaggcggccgtcatactccgttcttcaaaggctaccgtccgcagttctacttccgtactactgacgtgactggtaccatcgaactgccggaaggcgtagagatggtaatgccgggcgacaacatcaaaatggttgttaccctgatccacccgatcgcgatggacgacggtctgcgtttcgcaatccgtgaaggcggccgtaccgttggcgcgggcgttgtagctaaagttctgggcaagcttaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcttcatcaccatcaccatcactaactgtacaagtaagaatcccaactgagcgccggtcgctaccattaccaacttgtctggtgtcaaaaataataggcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - DNA, 3651 bp, Artificial Sequence
  Description of Artificial Sequence Domain 3 of EF-Tu-GFP-His of vector pEGFP
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagcgctaagccgggcaccatcaagccgcacaccaagttcgaatctgaagtgtacattctgtccaaagatgaaggcggccgtcatactccgttcttcaaaggctaccgtccgcagttctacttccgtactactgacgtgactggtaccatcgaactgccggaaggcgtagagatggtaatgccgggcgacaacatcaaaatggttgttaccctgatccacccgatcgcgatggacgacggtctgcgtttcgcaatccgtgaaggcggccgtaccgttggcgcgggcgttgtagctaaagttctgggctgcaagcttaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcttcatcaccatcaccatcactaactgtacaagtaagaatcccaactgagcgccggtcgctaccattaccaacttgtctggtgtcaaaaataataggcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 3 - DNA, 42 bp, Artificial Sequence
  Description of Artificial Sequence Synthetic oligonucleotide
gacgagctgtacaagtaaagcggccgcgactctagaattcca

SEQ ID NO: 4 - DNA, 44 bp, Artificial Sequence
  Description of Artificial Sequence Synthetic oligonucleotide
gtacaagcttcatcaccatcaccatcactaactgtacaagtaag

SEQ ID NO: 5 - DNA, 44 bp, Artificial Sequence
  Description of Artificial Sequence Synthetic oligonucleotide
ttcgaagtagtggtagtggtagtgattgacatgttcattcttaa

SEQ ID NO: 6 - protein, 9 aa, Artificial Sequence
  Description of Artificial Sequence Synthetic peptide
YKLHHHHHH

SEQ ID NO: 7 - DNA, 21 bp, Artificial Sequence
  Description of Artificial Sequence Synthetic oligonucleotide
actagctgcagcatgtctaaa

SEQ ID NO: 8 - protein, 7 aa, Artificial Sequence
  Description of Artificial Sequence Synthetic peptide
TSCSMSK

SEQ ID NO: 9 - DNA, 21 bp, Artificial Sequence
  Description of Artificial Sequence Synthetic oligonucleotide
ctgggcaagcttaccatggtg

SEQ ID NO: 10 - protein, 7 aa, Artificial Sequence
  Description of Artificial Sequence Synthetic peptide
LGKLTMV

SEQ ID NO: 11 - DNA, 21 bp, Artificial Sequence
  Description of Artificial Sequence Synthetic oligonucleotide
actagctgcagcgctaagccg

SEQ ID NO: 12 - protein, 7 aa, Artificial Sequence
  Description of Artificial Sequence Synthetic peptide
TSCSAKP

SEQ ID NO: 13 - DNA, 24 bp, Artificial Sequence
  Description of Artificial Sequence Synthetic oligonucleotide
ctgggctgcaagcttaccatggtg

SEQ ID NO: 14 - protein, 8 aa, Artificial Sequence
  Description of Artificial Sequence Synthetic peptide
LGCKLTMV